(19) United States
(12) Patent Application Publication
(10) Pub. No.: US 2022/0160260 A1
(43) Pub. Date: May 26, 2022

(54) SYSTEM AND METHOD FOR MEASURING BIOMEDICAL SIGNAL

(71) Applicant: ELECTRONICS AND TELECOMMUNICATIONS RESEARCH INSTITUTE, Daejeon (KR)

(72) Inventors: Moonwook RYU, Daejeon (KR); KWANGHYUN SHIM, Daejeon (KR); JUN SEONG LEE, Daejeon (KR); Hyuk JEONG, Daejeon (KR)

(21) Appl. No.: 17/524,819

(22) Filed: Nov. 12, 2021

(30) Foreign Application Priority Data
Nov. 25, 2020 (KR) 10-2020-0159997
Jun. 3, 2021 (KR) 10-2021-0072372

(51) Int. Cl. A61B 5/11; A61B 5/00; A61B 5/024

(52) U.S. Cl. CPC A61B 5/1128; A61B 5/0077; A61B 5/7246; A61B 5/0013; A61B 5/742; A61B 5/024; A61B 5/7264

(57) ABSTRACT

Disclosed is a biometric signal measuring system which includes an image obtaining unit that obtains an image of a user, an adaptive block module that combines biometric signals extracted from a face region based on the image provided from the image obtaining module and estimates a biometric signal of the user, and a biometric signal output module that outputs the biometric signal estimated from the adaptive block module to the outside.

### CROSS-REFERENCE TO RELATED APPLICATIONS

This application claims priority under 35 U.S.C. § 119 to Korean Patent Application Nos. 10-2020-0159997 filed on Nov. 25, 2020 and 10-2021-0072372 filed on Jun. 3, 2021, in the Korean Intellectual Property Office, the disclosures of which are incorporated by reference herein in their entireties.

### BACKGROUND

### 1. Field of the Invention

The present disclosure relates to a health care technology, and more particularly, relates to a system for measuring an image-based biometric signal by using an adaptive block module and a biometric signal measuring method thereof.

### 2. Description of Related Art

With the global aging trend, the importance of a health care technology that is used in medical fields such as a rehabilitation field and a health examination field is increasing. In particular, as the need for a vitro diagnostic service and a home health care service increases in the aftermath of COVID-19, the interest in monitoring technology using a biometric signal is increasing. Biometric signal data of the human may be utilized in various fields, and in particular, heart rate data of the biometric signal may be utilized to analyze the amount of exercise of a user, user's emotion, and the like.

Conventionally, the heart rate has been obtained by emitting a light of a specific wavelength to the user's finger or earlobe and detecting a change in a blood volume based on the intensity of a reflected or transmitted light. Because a heart rate measuring device based on the above scheme is implemented with a contact-type device capable of being in close contact with the user's skin, the heart rate measuring device has been mainly applied to wearable devices such as wrist watches and earphones. However, in the case of measuring the heart rate of the user through the wearable device, various noises may occur due to the movement and tension of the user, and thus, it is difficult to measure the heart rate more accurately.

To overcome the above issue, a non-contact way to measure a heart rate by using a camera image is proposed. According to the non-contact way to measure the heart rate, the heart rate of the user is obtained by recognizing the user's face through a camera image, setting a region of interest for analyzing a biometric signal, obtaining color change data, which are based on the heart rate, in the region of interest, and processing the obtained data. In the case of measuring the user's heart rate in the non-contact way, for accurate heart rate measurement, the user should not move his/her face while measuring his/her heart rate. However, because a noise occurs due to a minute movement or a facial expression change of the user in a daily living environment, there is a limitation in measuring a heart rate more accurately.

### SUMMARY

Embodiments of the present disclosure provide a system for measuring an image-based biometric signal in a non-contact manner by using an adaptive block module and a biometric signal measuring method thereof.

According to an embodiment, a biometric signal measuring system includes an image obtaining unit that obtains an image of a user, an adaptive block module that combines biometric signals extracted from a face region based on the image provided from the image obtaining module and estimates a biometric signal of the user, and a biometric signal output module that outputs the biometric signal estimated from the adaptive block module to the outside.

As an example, the image obtaining module includes an image obtaining unit that includes at least one photographing device and obtains the image of the user, and an image storing unit that stores the image of the user thus obtained.

As an example, when the image obtaining unit includes two or more photographing devices, the image obtaining unit performs a calibration operation for matching positions, resolutions, and fields of view of the photographing devices.

As an example, the adaptive block module includes a face region detecting unit that detects the face region by using the image of the user, a region weight calculating unit that calculates a weight of the face region detected from the face region detecting unit, a background region detecting unit that detects a background region other than the face region from the image of the user, a lighting change detecting unit that detects a lighting change of the background region, a biometric signal extracting unit that extracts the biometric signal of the face region based on the face region and the lighting change, and an ensemble correcting unit that estimates the biometric signal of the user by combining the biometric signal extracted from the face region and the weight of the face region.

As an example, the face region detecting unit performs a face detecting operation and a face landmark extracting operation, the face detecting operation is performed to transform image coordinates at which a face of the user is located in a bounding box shape, and the face landmark extracting operation is performed to estimate a face model of the user based on a standard face model and to separate the face region based on the face model thus estimated.

As an example, the face detecting operation is performed by a template matching technique or a deep learning-based face search technique.

As an example, the biometric signal output module includes a biometric signal information display unit that displays information about the biometric signal of the user estimated from the adaptive block module.

As an example, the biometric signal output module further includes a communication unit configured to send the information about the biometric signal of the user to the outside.

According to an embodiment, a biometric signal measuring method of a non-contact biometric signal measuring system for obtaining a heart rate of a user includes detecting a face region of the user by using an image of the user obtained from an image obtaining module, detecting a background region by using the image of the user, calculating a weight of the face region of the user, detecting a lighting change of the background region, extracting a biometric signal from the face region of the user based on the lighting change, and estimating the biometric signal of the user by combining the weight of the face region and the biometric signal extracted from the face region of the user.

As an example, the method further includes outputting information about the estimated biometric signal through a display device.

As an example, the method further includes sending information about the estimated biometric signal to the outside.

As an example, the detecting of the face region of the user includes transforming image coordinates at which a face of the user is located, estimating a face model of the user based on a standard face model, and separating the face region based on the face model of the user.

### BRIEF DESCRIPTION OF THE DRAWINGS

The above and other objects and features of the present disclosure will become apparent by describing in detail embodiments thereof with reference to the accompanying drawings.

FIG. 1 is a block diagram illustrating a biometric signal measuring system according to an embodiment of the present disclosure.

FIG. 2 is a diagram for describing an operation of an image obtaining module according to an embodiment of the present disclosure.

FIG. 3 is a block diagram for describing an operation of an adaptive block module according to an embodiment of the present disclosure.

FIG. 4 is a diagram for describing an operation of a biometric signal output module according to an embodiment of the present disclosure.

FIG. 5 is a flowchart for describing a biometric signal measuring method according to an embodiment of the present disclosure.

FIG. 6 is a flowchart for describing a method of estimating a biometric signal, in a biometric signal measuring method according to an embodiment of the present disclosure.

### DETAILED DESCRIPTION

Below, embodiments of the present disclosure will be described in detail and clearly to such an extent that one skilled in the art easily carries out the present disclosure.

The terms used in the specification are provided to describe the embodiments, not to limit the present disclosure. As used in the specification, the singular terms “a,” “an” and “the” are intended to include the plural forms as well, unless the context clearly indicates otherwise. The terms “comprises” and/or “comprising,” when used in the specification, specify the presence of steps, operations, elements, and/or components, but do not preclude the presence or addition of one or more other steps, operations, elements, components, and/or groups thereof.

In the specification, the term “first and/or second” will be used to describe various elements but will be described only for the purpose of distinguishing one element from another element, not limiting an element of the corresponding term. For example, without departing the scope of the present disclosure, a first element may be referred to as a second element, and similarly, a second element may be referred to as a first element.

Unless otherwise defined, all terms (including technical and scientific terms) used in the specification should have the same meaning as commonly understood by those skilled in the art to which the present disclosure pertains. The terms, such as those defined in commonly used dictionaries, should not be interpreted in an idealized or overly formal sense unless expressly so defined herein. The same reference numerals represent the same elements throughout the specification.

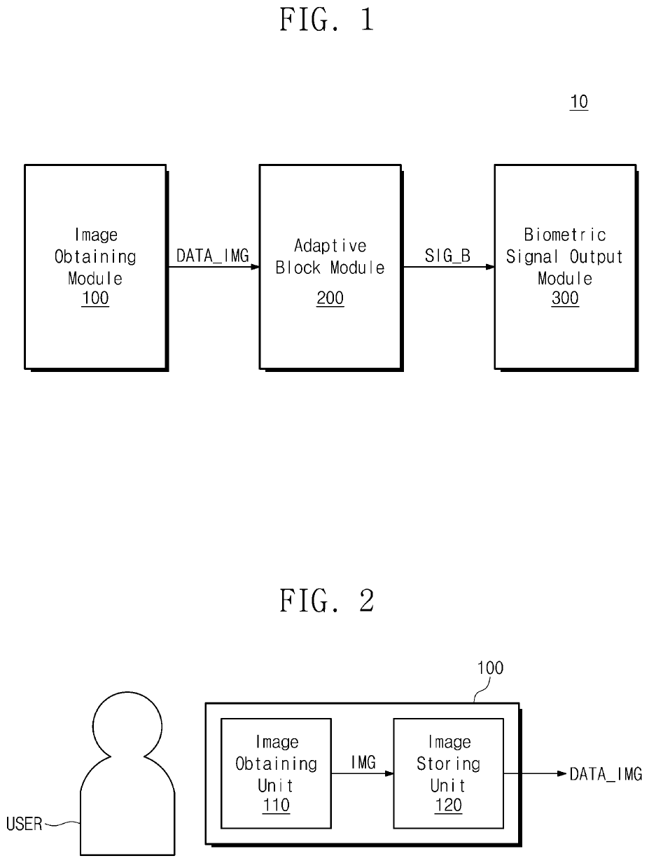

FIG. 1 is a block diagram illustrating a biometric signal measuring system 10 according to an embodiment of the present disclosure. Referring to FIG. 1, the biometric signal measuring system 10 according to an embodiment of the present disclosure may include an image obtaining module 100, an adaptive block module 200, and a biometric signal output module 300.

The image obtaining module 100 may obtain a face image of a user. The image obtaining module 100 may store the face image of the user thus obtained. The image obtaining module 100 may transfer face image data DATA_IMG stored therein to the adaptive block module 200. A configuration of the image obtaining module 100 will be described in detail with reference to FIG. 2.

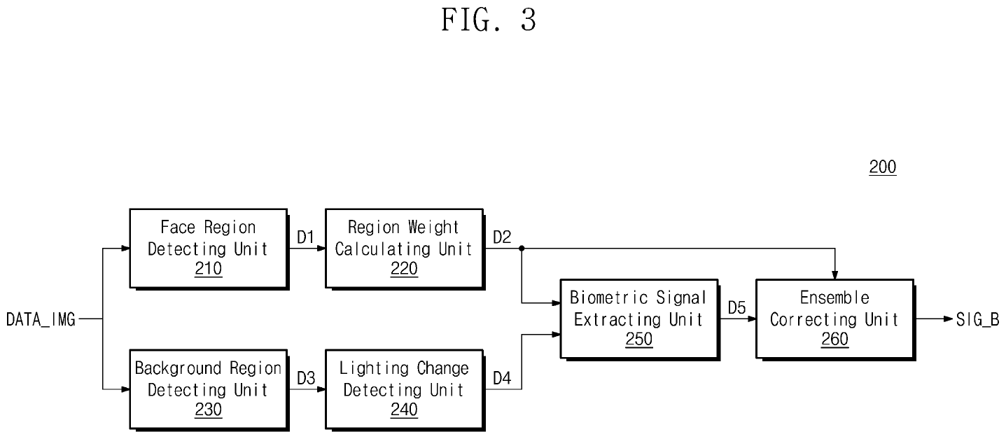

The adaptive block module 200 may estimate a biometric signal SIG_B of the user based on the face image data DATA_IMG received from the image obtaining module 100. For example, the biometric signal SIG_B of the user may indicate a heart rate. The adaptive block module 200 may remove an environmental noise capable of being created in a daily environment from the face image data DATA_IMG. For example, the environmental noise may refer to a noise caused by a movement of the user, a change in facial expression of the user, self-occlusion, a change in ambient lighting, and the like. After removing the environmental noise, the adaptive block module 200 may estimate the biometric signal SIG_B of the user and may transfer the biometric signal SIG_B to the biometric signal output module 300. A configuration of the adaptive block module 200 will be described in detail with reference to FIG. 3.

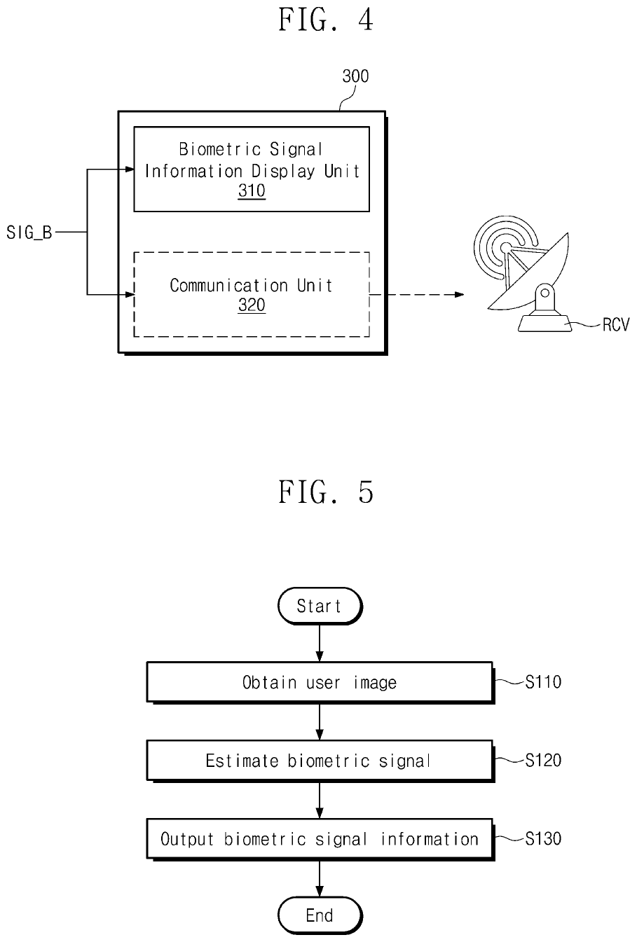

The biometric signal output module 300 may output information about the biometric signal SIG_B of the user received from the adaptive block module 200. A configuration of the biometric signal output module 300 will be described in detail with reference to FIG. 4.

The biometric signal measuring system 100 according to an embodiment of the present disclosure may remove various environmental noises capable of being created in a daily life through the adaptive block module 200 and may then extract the biometric signal SIG_B of the user. As such, the biometric signal measuring system 100 may obtain the biometric signal SIG_B of the user more accurately. The biometric signal measuring system 10 according to an embodiment of the present disclosure may be used to automatically monitor a body state of the user. Also, the biometric signal measuring system 10 according to an embodiment of the present disclosure may send information about the measured biometric signal SIG_B so as to be used in a telemedicine system. Also, the biometric signal measuring system 10 according to an embodiment of the present disclosure may be used in a system that analyzes an emotional state of the user based on the measured biometric signal SIG_B and provides a variety of content based on an analysis result.

FIG. 2 is a diagram for describing an operation of the image obtaining module 100 according to an embodiment of the present disclosure. The image obtaining module 100 may generate the face image data DATA_IMG for measuring a biometric signal of the user “USER”. Referring to FIG. 2, the image obtaining module 100 may include an image obtaining unit 110 and an image storing unit 120.

The image obtaining unit 110 may obtain a face image IMG of the user “USER”. For example, the face image IMG of the user “USER” may be a two-dimensional color image associated with the user “USER” or a three-dimensional image associated with the user “USER”.

The image obtaining unit 110 may include at least one photographing device. For example, the at least one photographing device may include a device, which obtains a two-dimensional color image, such as a webcam or a camera module of a mobile device, or a depth camera, which obtains a three-dimensional image, such as a stereo camera or a kinect. In the case where the image obtaining unit 110 includes a plurality of photographing devices, the image obtaining unit 110 may perform a calibration operation for matching positions, resolutions, and fields of view of the photographing devices. The calibration operation may include performing affine transformation on coordinates of the remaining photographing devices based on one photographing device. Meanwhile, in the case where the image obtaining unit 110 includes a single photographing device, the calibration operation may be omitted. The image obtaining unit 110 may transfer the face image IMG of the user “USER” thus obtained to the image storing unit 120.

The image storing unit 120 may store the face image IMG of the user “USER” received from the image obtaining unit 110. The image storing unit 120 may transfer the face image data DATA_IMG, which are based on the face image IMG of the user “USER”, to the adaptive block module 200 (refer to FIG. 1).

FIG. 3 is a block diagram for describing an operation of the adaptive block module 200 according to an embodiment of the present disclosure. The adaptive block module 200 may obtain the biometric signal SIG_B of the user after removing an environmental noise from the face image data DATA_IMG received from the image obtaining module 100. Referring to FIG. 3, the adaptive block module 200 may include a face region detecting unit 210, a region weight calculating unit 220, a background region detecting unit 230, a lighting change detecting unit 240, a biometric signal extracting unit 250, and an ensemble correcting unit 260.

The face region detecting unit 210 may receive the face image data DATA_IMG and may detect a face region of the user “USER” (refer to FIG. 2), from which the biometric signal SIG_B is to be measured, from the face image data DATA_IMG. The detection of the face region may be performed through a face detecting operation and a face landmark extracting operation. The face detecting operation that refers to the process of setting a region of interest for extracting a face landmark may be performed by transforming image coordinates at which a face is located in a bounding box shape. For example, the face detecting operation may be performed in various schemes such as a template matching technique and a deep learning-based face search technique.

The face landmark extracting operation may include setting a calculation region for sensing (or detecting) a movement or a change in the facial expression of the user “USER” or constructing ensemble data. The face landmark extracting operation may be performed in the process of separating a face region after a face model of the user “USER” is estimated based on a standard face model. The face landmark may be output by using a coordinate value based on the standard face model. For example, in the case of using a two-dimensional standard face model, a two-dimensional landmark coordinate value may be output; in the case of using a three-dimensional standard face model, a three-dimensional landmark coordinate value may be output. According to the face landmark extracting operation of the present disclosure, a face landmark may not be extracted every frame, but each face region may be extracted after a face model of the user is estimated.

Accordingly, a noise that comes from a change in the face scale according to the front and back movement of a photographing device and a face movement according to up/down/left/right rotations of the face may be minimized. The face region detecting unit 210 may generate first data D1 indicating the face region of the user obtained after the face detecting operation and the face landmark extracting operation are performed, and may transfer the first data D1 to the region weight calculating unit 220.

The region weight calculating unit 220 may receive the first data D1 from the face region detecting unit 210 and may calculate a weight for each time by using the first data D1. In detail, based on the first data D1, the region weight calculating unit 220 may calculate a weight for each time associated with a change of a face region, which is variable along a movement in a daily life. The weight for each time means a reliability of each region in a current frame phase. For example, the movement in a daily life may include a change in the face expression that includes 1) a change in the face expression according to an occlusion situation or emotion, which occurs instantaneously, such as a face region, hairs, a face movement, or a hand movement that is the most visible at a current photographing point in time, and 2) a change in a region around the mouth during conversation; when a movement capable of occurring in a daily life occurs, a weight may be expressed low. The region weight calculating unit 220 may generate second data D2 that include a weight for each time associated with a change in a face region, and may provide the second data D2 to the biometric signal extracting unit 250 and the ensemble correcting unit 260.

The background region detecting unit 230 may extract a region other than the face region for the purpose of removing a noise according to a change in the intensity of an ambient light, when measuring the biometric signal SIG_B. For example, in the case of using a two-dimensional image obtained from a color camera, a face (or face region) of the user and a background region may be segmented based on a machine learning technique. Alternatively, in the case of using a three-dimensional image obtained from a depth camera, a background region may be extracted by using a depth value. The background region detecting unit 230 may set regions of an object having a high reflectance of well reflecting an ambient luminance environment and an object according to a color or a reflectance, not extracting all the remaining regions other than a face region. Third data D3 indicating the object region set by the background region detecting unit 230 may be transferred to the lighting change detecting unit 240.

The lighting change detecting unit 240 may obtain a lighting intensity for each region of the object based on the third data D3 received from the background region detecting unit 230. In this case, because an object region is set, even when a photographing device moves, a change in an ambient lighting intensity may be sensed. The lighting change detecting unit 240 may extract the trend of a brightness change of the whole image based on the obtained lighting intensity and may remove a noise according to a lighting change based on the extracted trend. The lighting change detecting unit 240 may generate fourth data D4 that are obtained by removing the noise according to the lighting change and may provide the fourth data D4 to the biometric signal extracting unit 250.

The biometric signal extracting unit 250 may receive the second data D2 and the fourth data D4 and may extract a biometric signal by filtering, for each face region, a signal associated with the biometric signal from a brightness change signal according to a time. For example, the biometric signal extracting unit 250 may transform the brightness change signal of each face region to a frequency domain through the Fourier transform, may select a peak value in a frequency range of a heart rate, and may extract a heart rate of the user “USER” by transforming the peak value to a time domain. Alternatively, a heart rate of the user may be extracted by transforming filtering data from a frequency domain of a heart rate to a time domain through the inversion Fourier transform such that a heartbeat signal is extracted in the time domain and selecting a peak value. Fifth data D5 indicating the biometric signal extracted from the biometric signal extracting unit 250 may be provided to the ensemble correcting unit 260.

The ensemble correcting unit 260 may receive the second data D2 and the fifth data D5, may respectively apply weights to face regions, and may combine biometric signals extracted from the face regions. Because the biometric signal extracted from each face region has a unique signal pattern for each face region, the ensemble correcting unit 260 may combine the biometric signals to estimate the biometric signal SIG_B of the user “USER”. The estimated biometric signal SIG_B may be provided to the biometric signal output module 300.

FIG. 4 is a diagram for describing an operation of the biometric signal output module 300 according to an embodiment of the present disclosure. The biometric signal output module 300 may output the biometric signal SIG_B estimated from the adaptive block module 200 (refer to FIG. 1) to the outside. Referring to FIG. 4, the biometric signal output module 300 may include a biometric signal information display unit 310, and may further include a communication unit 320 if necessary.

The biometric signal information display unit 310 may include a device that outputs the biometric signal SIG_B measured from the face image of the user “USER” (refer to FIG. 2). For example, the biometric signal information display unit 310 may include a display device such as a projector or a monitor. Biometric signal information based on the measured biometric signal SIG_B may be displayed through the display device such that the user “USER” performs self-monitoring on a body state.

The communication unit 320 may send the biometric signal SIG_B measured from the face image of the user “USER” (refer to FIG. 2) to the outside. For example, an external receiver RCV may receive the biometric signal SIG_B sent from the communication unit 320 and may use the biometric signal SIG_B in a telemedicine system.

FIG. 5 is a flowchart for describing a biometric signal measuring method according to an embodiment of the present disclosure.

In operation S110, the biometric signal measuring system 10 (refer to FIG. 1) according to an embodiment of the present disclosure may obtain a face image of the user “USER” (refer to FIG. 2). The biometric signal measuring system 10 according to an embodiment of the present disclosure may store the face image of the user “USER” thus obtained.

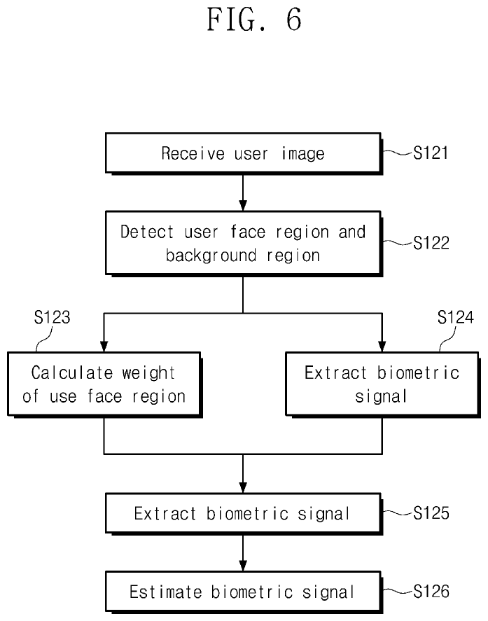

In operation S120, the biometric signal measuring system 10 according to an embodiment of the present disclosure may estimate a biometric signal of the user “USER” from the face image of the user “USER”. The biometric signal measuring system 10 according to an embodiment of the present disclosure may estimate a biometric signal from which a noise created due to a movement of the user “USER”, a change of facial expression of the user “USER”, or a change in ambient luminance is removed, which will be described with reference to FIG. 6.

In operation S130, the biometric signal measuring system 10 according to an embodiment of the present disclosure may output biometric signal information to the outside based on the estimated biometric signal of the user “USER”. For example, the biometric signal information may be displayed to the user “USER” through the display device or may be sent to an external server.

FIG. 6 is a flowchart for describing a method of estimating a biometric signal, in a biometric signal measuring method according to an embodiment of the present disclosure.

In operation S121, the biometric signal measuring system 10 (refer to FIG. 1) according to an embodiment of the present disclosure may receive an image of the user “USER” (refer to FIG. 2) obtained from the image obtaining module 100 (refer to FIG. 1).

In operation S122, the adaptive block module 200 may detect a face region of the user “USER” and a background region based on the image of the user “USER” thus obtained. The face region of the user “USER” may be detected through the face detecting operation and the face landmark extracting operation. The background region may be detected through the operation of extracting a region of an object having high reflectance of well reflecting an ambient luminance environment and a region of an object according to a color or a reflectance.

In operation S123, the adaptive block module 200 may calculate a weight of a face region by using the face region detected in operation S122. In detail, the adaptive block module 200 may calculate a weight for each time associated with a change of a face region, which is variable depending on a movement in a daily life.

In operation S124, the adaptive block module 200 may detect a lighting change of the background region by using the background region detected in operation S122. The adaptive block module 200 may extract the trend of a brightness change of the whole image based on an obtained lighting intensity and may remove a noise according to a lighting change based on the extracted trend.

In operation S125, the adaptive block module 200 may extract a biometric signal by filtering, for each face region, a signal associated with the biometric signal from a brightness change signal according to a time.

In operation S126, the adaptive block module 200 may estimate a biometric signal of the user “USER” by combining the weight for each time calculated in operation S123 and a biometric signal extracted from each face region extracted in operation S125.

In some embodiments, the biometric signal measuring method according to an embodiment of the present disclosure described with reference to FIGS. 5 and 6 may be implemented in the form of a program instruction executable through various computing devices and may be recorded in a computer-readable medium. The computer-readable medium may include program instructions, data files, data structures, and the like independently or may include a combination thereof. The program instruction(s) recorded in the computer-readable medium may be specially designed and configured for the present disclosure or may be known and available to those skilled in computer software.

For example, the computer-readable medium may include a hardware device, which is configured to store and execute program instructions, such as magnetic media (e.g., a hard disk drive and a magnetic tape), optical recording media (e.g., CD-ROM and DVD), magneto-optical media (e.g., a floptical disk), read only memories (ROMs), random access memories (RAMs), and flash memories. For example, the program instruction may include high-level language code executable by a computer using an interpreter or the like, as well as machine code generated by a compiler, and the hardware device may be configured to operate through one or more software modules for performing the operation of the present disclosure.

According to the present disclosure, in a system and a method for measuring a biometric signal, various environmental noises are removed through an adaptive block module, and thus, biometric signal data of the user are obtained more accurately.

While the present disclosure has been described with reference to embodiments thereof, it will be apparent to those of ordinary skill in the art that various changes and modifications may be made thereto without departing from the spirit and scope of the present disclosure as set forth in the following claims.

What is claimed is:

1. A biometric signal measuring system comprising:
an image obtaining unit configured to obtain an image of a user;
an adaptive block module configured to combine biometric signals extracted from a face region based on the image provided from the image obtaining module and to estimate a biometric signal of the user; and
a biometric signal output module configured to output the biometric signal estimated from the adaptive block module to the outside.

2. The biometric signal measuring system of claim 1, wherein the image obtaining module includes:
an image obtaining unit including at least one photographing device, and configured to obtain the image of the user; and
an image storing unit configured to store the image of the user thus obtained.

3. The biometric signal measuring system of claim 2, wherein, when the image obtaining unit includes two or more photographing devices, the image obtaining unit performs a calibration operation for matching positions, resolutions, and fields of view of the photographing devices.

4. The biometric signal measuring system of claim 1, wherein the adaptive block module includes:
a face region detecting unit configured to detect the face region by using the image of the user;
a region weight calculating unit configured to calculate a weight of the face region detected from the face region detecting unit;
a background region detecting unit configured to detect a background region other than the face region from the image of the user;
a lighting change detecting unit configured to detect a lighting change of the background region;
a biometric signal extracting unit configured to extract the biometric signal of the face region based on the face region and the lighting change; and
an ensemble correcting unit configured to estimate the biometric signal of the user by combining the biometric signal extracted from the face region and the weight of the face region.

5. The biometric signal measuring system of claim 4, wherein the face region detecting unit performs a face detecting operation and a face landmark extracting operation,
wherein the face detecting operation is performed to transform image coordinates at which a face of the user is located in a bounding box shape, and
wherein the face landmark extracting operation is performed to estimate a face model of the user based on a standard face model and to separate the face region based on the face model thus estimated.

6. The biometric signal measuring system of claim 5, wherein the face detecting operation is performed by a template matching technique or a deep learning-based face search technique.

7. The biometric signal measuring system of claim 1, wherein the biometric signal output module includes:
a biometric signal information display unit configured to display information about the biometric signal of the user estimated from the adaptive block module.

8. The biometric signal measuring system of claim 7, wherein the biometric signal output module further includes:
a communication unit configured to send the information about the biometric signal of the user to the outside.

9. A biometric signal measuring method of a non-contact biometric signal measuring system for obtaining a heart rate of a user, the biometric signal measuring method comprising:
detecting a face region of the user by using an image of the user obtained from an image obtaining module;
detecting a background region by using the image of the user;
calculating a weight of the face region of the user;
detecting a lighting change of the background region;
extracting a biometric signal from the face region of the user based on the lighting change; and
estimating the biometric signal of the user by combining the weight of the face region and the biometric signal extracted from the face region of the user.

10. The biometric signal measuring method of claim 9, further comprising:
outputting information about the estimated biometric signal through a display device.

11. The biometric signal measuring method of claim 9, further comprising:
sending information about the estimated biometric signal to the outside.

12. The biometric signal measuring method of claim 9, wherein the detecting of the face region of the user includes:
transforming image coordinates at which a face of the user is located;
estimating a face model of the user based on a standard face model; and
separating the face region based on the face model of the user.
